(19) United States
(12) Patent Application Publication
(10) Pub. No.: US 2025/0073085 A1
(43) Pub. Date: Mar. 6, 2025

(54) ELASTIC BANDAGE

(71) Applicants: Hung-Yang Chen, New Taipei (TW); Hsiang-Yu Chen, New Taipei (TW)

(72) Inventors: Hung-Yang Chen, New Taipei (TW); Hsiang-Yu Chen, New Taipei (TW)

(21) Appl. No.: 18/241,239

(22) Filed: Sep. 1, 2023

(51) Int. Cl. A61F 13/00

(52) U.S. Cl. CPC A61F 13/01038

(57) ABSTRACT

An elastic bandage includes: a body including a wide portion and a narrow portion, where the wide portion is connected to the narrow portion along an X-axis direction, and along a Y-axis direction perpendicular to the X-axis direction, a width of the wide portion is greater than a width of the narrow portion; a first fixing portion disposed on the wide portion of the body; and a second fixing portion disposed on the narrow portion of the body; where the first fixing portion and the second fixing portion are respectively located at two opposite ends of the body along the X-axis direction.

### FIELD OF TECHNOLOGY

The present invention relates to medical supplies, and in particular to an elastic bandage with different width ratios and a reinforced fixing function.

### BACKGROUND

The elastic bandage is a common medical supply and widely applied to sports protection and injury rehabilitation. It is a soft and elastic textile that can provide mild compression and support while maintaining joint mobility.

Being elastic and flexible is one of the main advantages of elastic bandage. The bandage being elastic allows it to be closely attached to the contour of the body, providing mild compression and support. It can also be stretchable and retractable, maintaining an appropriate level of snugness with the movement of muscles and joints, thereby increasing the comfort during physical activities. In addition, the elastic bandage is breathable and can wick sweat. The bandage is typically made from a textile material that features breathability, facilitating heat dissipation and preventing excessive sweating. The bandage is crucial for long-term wear and comfort during physical activities and is less likely to slip or roll.

However, despite the numerous advantages of commonly used elastic bandages, there are still some issues that need to be known and considered regarding their usage. For example, the commonly used elastic bandage has a fixed width, which is relatively large, to achieve better coverage. Consequently, when wrapped around the affected part, the bandage may encounter some obstacles, leading to fixing difficulties. Moreover, the fixed width or length can result in excessive coverage, affecting the mobility of the affected part.

Additionally, the commonly used elastic bandage typically has a fixed structure, with one end adhered to the other end along its length direction. The bandage, when used as a single fixed structure, is likely to come off when the affected part experiences a slightly intense movement, and thus needs to be refixed, causing inconvenience during use.

Therefore, it is urgent for those in the related field to figure out how to elevate the problem about wrapping and fixing the commonly used elastic bandage.

### SUMMARY

A main objective of the present invention is to provide an elastic bandage able to provide a simpler and more stable experience of bandage use.

To achieve the foregoing invention objective, the present invention provides an elastic bandage, including: a body including a wide portion and a narrow portion, where the wide portion is connected to the narrow portion along an X-axis direction, and along a Y-axis direction perpendicular to the X-axis direction, a width of the wide portion is greater than a width of the narrow portion; a first fixing portion disposed on the wide portion of the body; and a second fixing portion disposed on the narrow portion of the body; where the first fixing portion and the second fixing portion are respectively located at two opposite ends of the body along the X-axis direction.

In an embodiment, along the X-axis direction, the wide portion is longer than the narrow portion.

In an embodiment, the width of at least part of the body gradually narrows along the Y-axis direction from the wide portion to the narrow portion.

In an embodiment, the first fixing portion has an opening in a predetermined shape and a movable piece at least partially connected to the wide portion and corresponding to the opening. The movable piece is drivable to move with respect to the opening along a Z-axis direction perpendicular to the X-axis direction and the Y-axis direction.

In an embodiment, the opening is in a shape of water droplet.

In an embodiment, the opening is in a semi-circular shape.

In an embodiment, the opening is in an X shape.

In an embodiment, the opening is in a V shape.

In an embodiment, the opening is in a A shape.

Further, in addition to the foregoing water droplet shape, semi-circular shape, X shape, V shape, or A shape, the predetermined shape may be another geometric shape or irregular shape.

In an embodiment, the first fixing portion further has a tangent with one end connected to the opening and the other end extending along the x-axis direction.

In an embodiment, the elastic bandage further includes an accommodating portion disposed on the wide portion of the body, where the body is provided with an accommodating space formed between the accommodating portion and the wide portion.

In an embodiment, the accommodating portion and the second fixing portion are respectively disposed on two opposite sides of the body along a Z-axis direction perpendicular to the X-axis direction and the Y-axis direction.

In an embodiment, a side of the accommodating portion is provided with an open end communicating with the accommodating space and configured for a filling material to enter and exit the accommodating space along the open end.

In an embodiment, two closed ends are provided on two opposite sides of the accommodating portion along the Y-axis direction and respectively connected to two opposite sides of the wide portion along the Y-axis direction; and two opposite sides of the accommodating portion along the X-axis direction are provided with two open ends communicating with the accommodating space and configured for a filling material to enter and exit the accommodating space along any one of the two open ends.

In an embodiment, the elastic bandage further includes an electrotherapy portion disposed between the accommodating portion and the wide portion and electrically connected to a power supply, so as to provide a microcurrent for an affected part when a user wraps the body around the affected part.

In an embodiment, the elastic bandage further includes a thermotherapy portion disposed between the accommodating portion and the wide portion and electrically connected to a power supply, so as to provide thermal energy for the affected part when the user wraps the body around the affected part.

In an embodiment, along the X-axis direction, the wide portion of the body is longer than the narrow portion.

In an embodiment, an elastic bandage includes: two bodies each including a wide portion and a narrow portion, where the wide portion and the narrow portion of each body are connected along an X-axis direction, and along a Y-axis direction perpendicular to the X-axis direction, a width of the wide portion is greater than a width of the narrow portion; two first fixing portions respectively disposed on the two wide portions of the two bodies; and two second fixing portions respectively disposed on the two narrow portions of the two bodies. The two wide portions of the two bodies are connected along the Y-axis direction.

In an embodiment, the two narrow portions are spaced apart from each other by a predetermined distance along the Y-axis direction.

In an embodiment, the elastic bandage further includes two accommodating portions respectively disposed on the two wide portions of the two bodies, the two accommodating portions being connected along the Y-axis direction.

Preferred embodiments are provided according to the objective, effects, and structural configurations disclosed by the present invention, and are described in detail with reference to accompanying drawings.

### BRIEF DESCRIPTION OF THE DRAWINGS

FIG. 1 is an external view according to a first preferred embodiment of the present invention.

FIG. 2 is a side view according to a first preferred embodiment of the present invention.

FIG. 3 is a schematic diagram according to a first preferred embodiment of the present invention, showing a state of the present invention wrapped around a hand of a user.

FIG. 4 is an external view according to a second preferred embodiment of the present invention.

FIG. 5 is an external view according to a third preferred embodiment of the present invention.

FIG. 6 is an external view according to a fourth preferred embodiment of the present invention.

FIG. 7 is an external view according to a fifth preferred embodiment of the present invention.

FIG. 8 is an external view according to a sixth preferred embodiment of the present invention.

FIG. 8A is an external view according to a seventh preferred embodiment of the present invention.

FIG. 8B is an external view according to an eighth preferred embodiment of the present invention.

FIG. 9 is an external view according to a ninth preferred embodiment of the present invention.

FIG. 10 is a schematic diagram according to the ninth preferred embodiment of the present invention, showing a state of filling an accommodating portion with a filling material.

FIG. 11 is an external view according to a tenth preferred embodiment of the present invention.

FIG. 12 is an external view according to an eleventh preferred embodiment of the present invention.

FIG. 13 is an external view according to a twelfth preferred embodiment of the present invention.

FIG. 14 is an external view according to a thirteenth preferred embodiment of the present invention.

Reference numerals are described as follows: 10. body; 11. wide portion; 12. narrow portion; 13. first end; 14. second end; 20. first fixing portion; 21. opening; 22. movable piece; 23. tangent; 30. second fixing portion; 31. velcro; 40. accommodating portion; 41. closed end; 42. open end; 50. accommodating space; 60. filling material; 61. electrotherapy portion; 62. thermotherapy portion; 100. affected part; 70. accommodating portion; 71. closed end; 72. open end; 73. accommodating space; 74. filling material; 80. body; 81. first fixing portion; 82. second fixing portion; 83. accommodating portion; 84. wide portion; and 85. narrow portion.

### DETAILED DESCRIPTION OF THE EMBODIMENTS

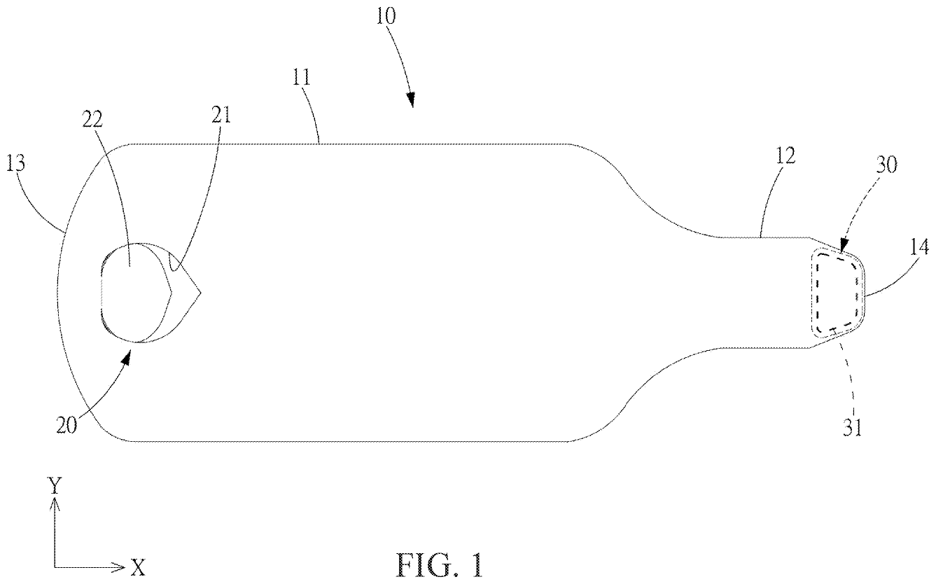

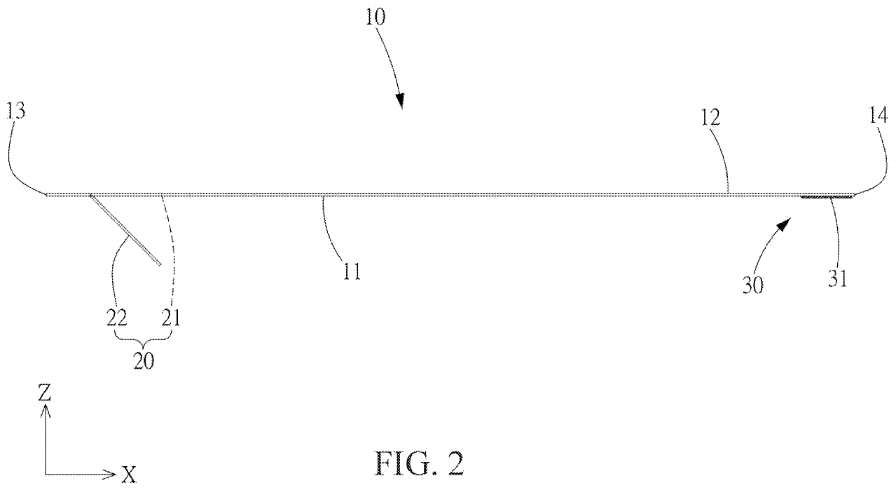

As shown in FIGS. 1 and 2, an elastic bandage disclosed by a first preferred embodiment of the present invention mainly includes: a body 10, a first fixing portion 20, and a second fixing portion 30. The body 10 is made of a non-woven fabric that is wine bottle-shaped, somewhat elastic, and waterproof, and includes a wide portion 11 and a narrow portion 12.

An X-axis direction is defined as the length direction of the body 10; a Y-axis direction is defined as the width direction of the body 10; and a Z-axis direction is defined as the thickness direction of the body 10. The X-axis, the Y-axis, and the Z-axis are mutually orthogonal (or perpendicular) to each other.

The wide portion 11 is connected to the narrow portion 12 along the X-axis direction. Along the X-axis direction, the wide portion 11 is longer than the narrow portion 12. Along the Y-axis direction perpendicular to the X-axis direction, the wide portion 11 is wider than the narrow portion 12. And, the width of at least part of the body 10 narrows along the Y-axis direction from the wide portion 11 to the narrow portion 12.

The two opposite ends of the body 10 along the X-axis direction respectively have a first end 13 and a second end 14. The first end 13 is adjacent to the wide portion 11 and generally arc-shaped. The second end 14 is adjacent to the narrow portion 12 and generally trapezoidal. The first fixing portion 20 is disposed on the wide portion 11 and adjacent to the first end 13 of the body 10, and the second fixing portion 14 is disposed on the narrow portion 12 and adjacent to the second end 14 of the body 10.

The first fixing portion 20 is provided with an opening 21 and a movable piece 22. The opening 21 is a hole enclosed by a secant in a predetermined shape. The movable piece 22 is a piece formed by part of the wide portion 11 separated along the secant to correspond to the opening 21. At least part of the movable piece 22 is connected to the wide portion 11 and movable with respect to the wide portion 11. The movable piece 22 can be driven by a user to move with respect to the opening 21 along the Z-axis direction perpendicular to the X-axis direction and Y-axis direction.

In this embodiment, the opening 21 and the movable piece 22 of the first fixing portion 20 are both in a shape of water drop. The second fixing portion 30 is provided with a velcro 31 disposed on a side of the body 10 along the Z-axis direction. The velcro 31 of the second fixing portion 30 can be adhered to any part of the body 10. In another embodiment, the second fixing portion can be configured with any other equivalent combination structure that can be fixed to the body.

In still another embodiment, the first fixing portion 20 is attached with a velcro (not shown in the figure). The movable piece 22 is also provided with a velcro (not shown in the figure). In this way, an adhesion state is enabled using the velcro 31, the first fixing portion 20, or the movable piece 22, thus enhancing the strength of fixing the body 10 to the affected part 100.

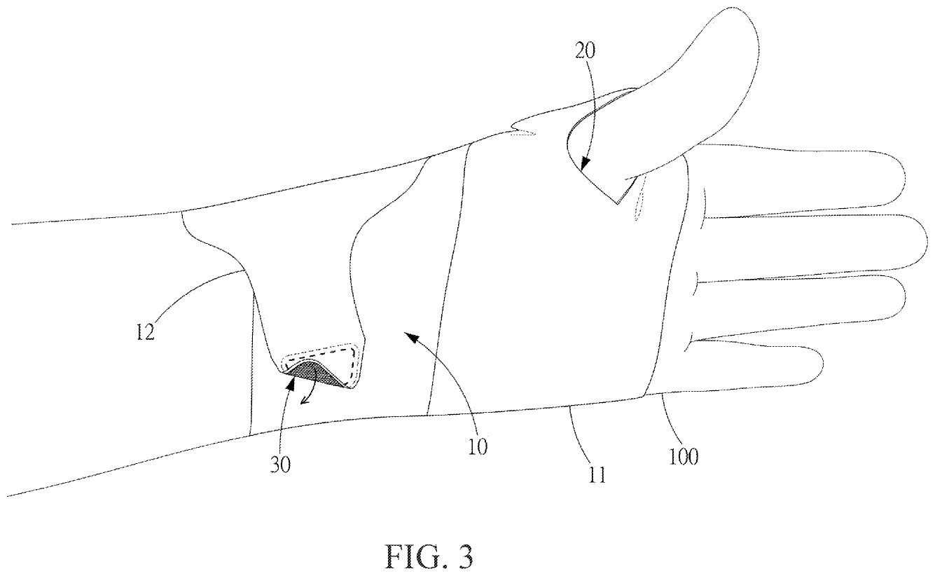

Referring to FIG. 3, when the user uses the present invention to bandage an affected part 100 (for example, the wrist in the figure), the fingers of the user can pass through the body 10 along the first fixing portion 20 and makes the body against the palm, then the body 10 is repeatedly wound around the affected part 100, and last the second fixing portion 30 is adhered to any part of the body 10. The first fixing portion 20 and the second fixing portion 30 being designed so can enhance the strength of fixing the body 10 to the affected part 100.

In addition, the design of different width ratios and length ratios between the wide portion 11 and the narrow portion 12 allows the body 10 to be wrapped more easily around the affected part 100, thus reducing the ineffective wrapping area. In another embodiment, the user can repeatedly wrap the body around the affected part, and then make the second fixing portion 30 penetrate through the first fixing portion 20 to be adhered to any part of the body. In this way. the first fixing portion 20 is used to enhance the fixing strength of the second fixing portion 30.

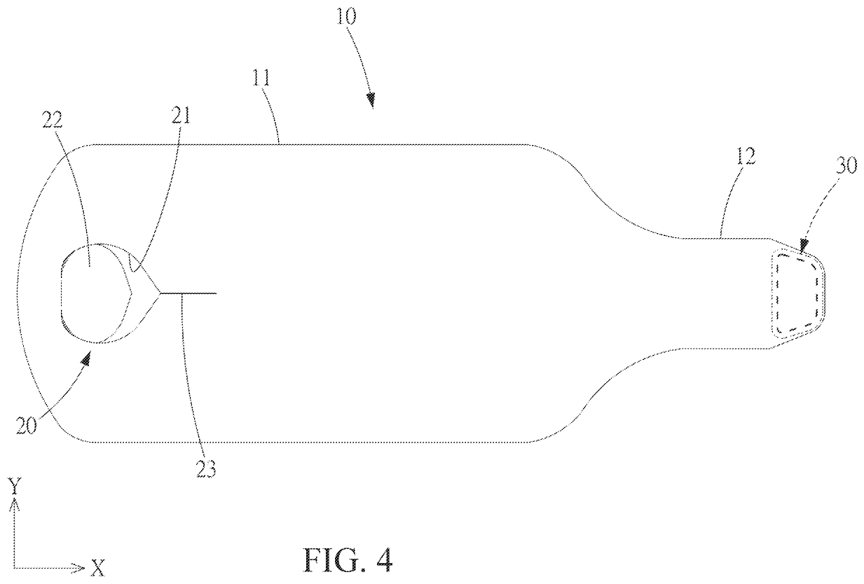

Referring to FIG. 4, the elastic bandage disclosed by a second preferred embodiment of the present invention has a structure similar to that of the elastic bandage in the first preferred embodiment. The first fixing portion 20 further has a tangent 23 with one end connected to an end of the opening 21 and the other end extending along the X-axis direction. In this way, when the user needs a larger opening 21, the wide portions 11 on two sides can be separated along the tangent 23, thus enlarging the area of the opening 21.

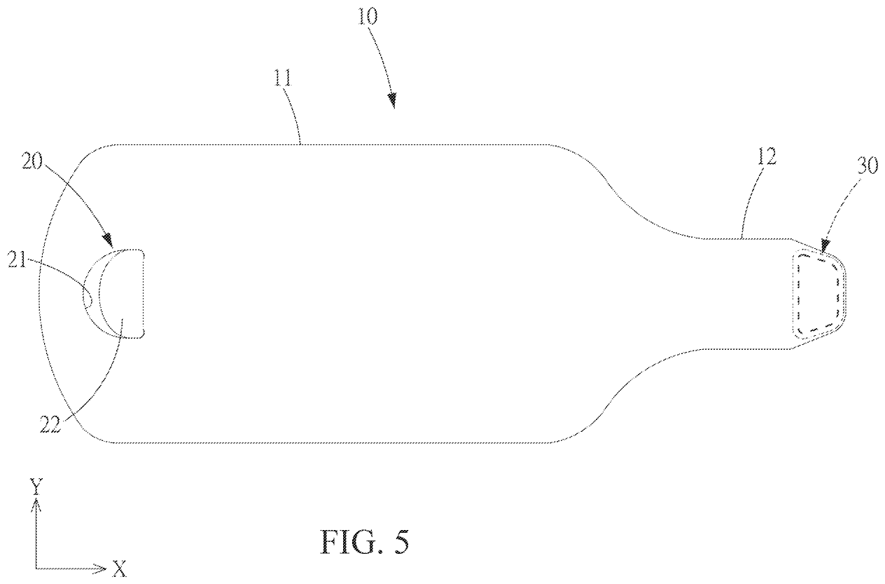

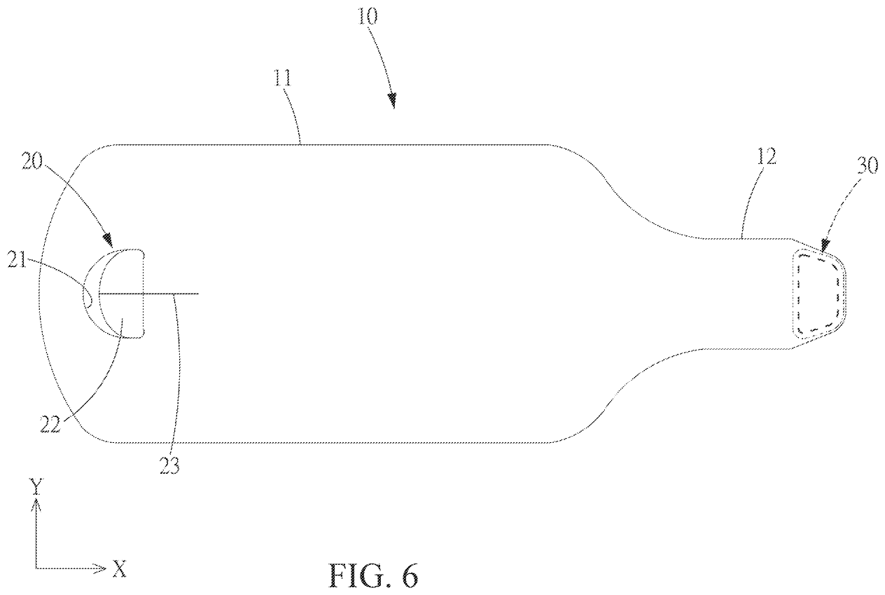

Referring to FIGS. 5 and 6, elastic bandages disclosed by a third preferred embodiment and a fourth embodiment of the present invention have structures similar to those of the elastic bandages in the foregoing embodiment. The opening 21 and the movable piece 22 of the first fixing portion 20 are half circular.

As compared with the third preferred embodiment, the first fixing portion 20 that is in the fourth preferred embodiment and similar to that in the second preferred embodiment, further has the tangent 23 extending along the X-axis direction, where the tangent has one end connected to an end of the opening 21 and the other end extending along the X-axis direction.

In this embodiment, the tangent 23 further divides the movable piece 22 into two pieces, each resembling a quarter of a circle. This not only increases the area of the opening 21 but also enhances the mobility of the movable piece 22.

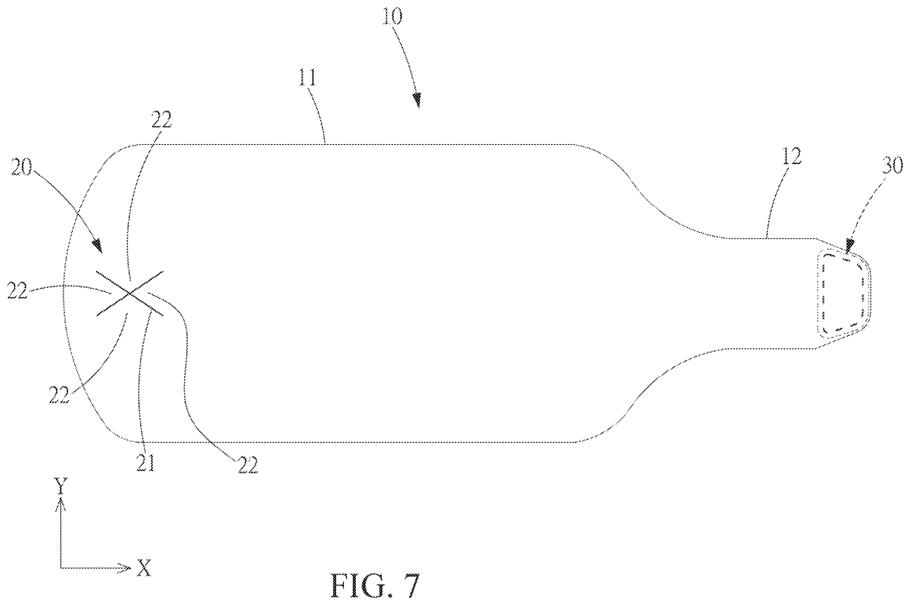

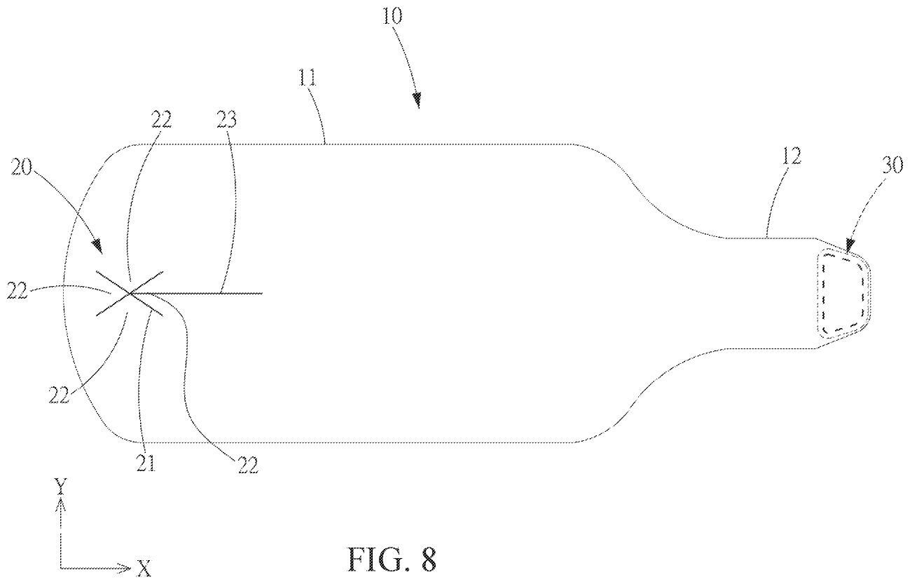

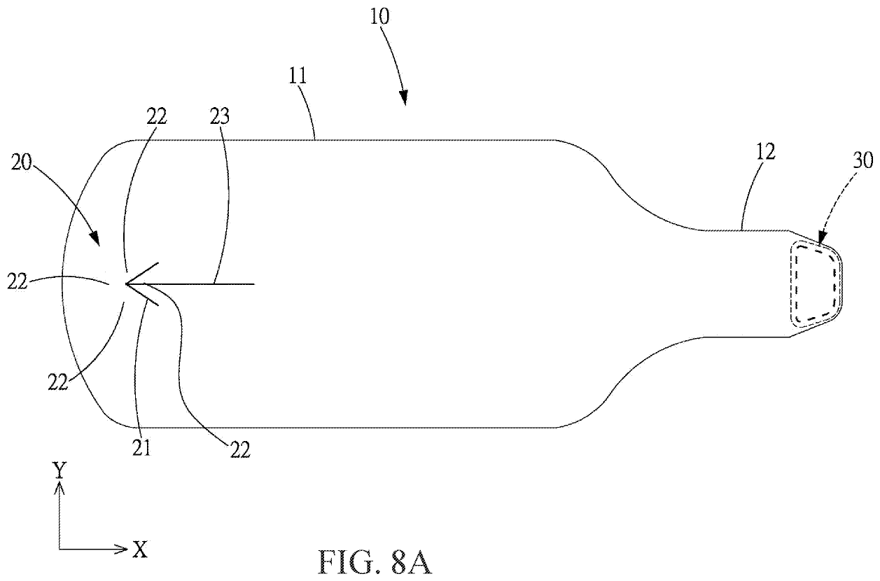

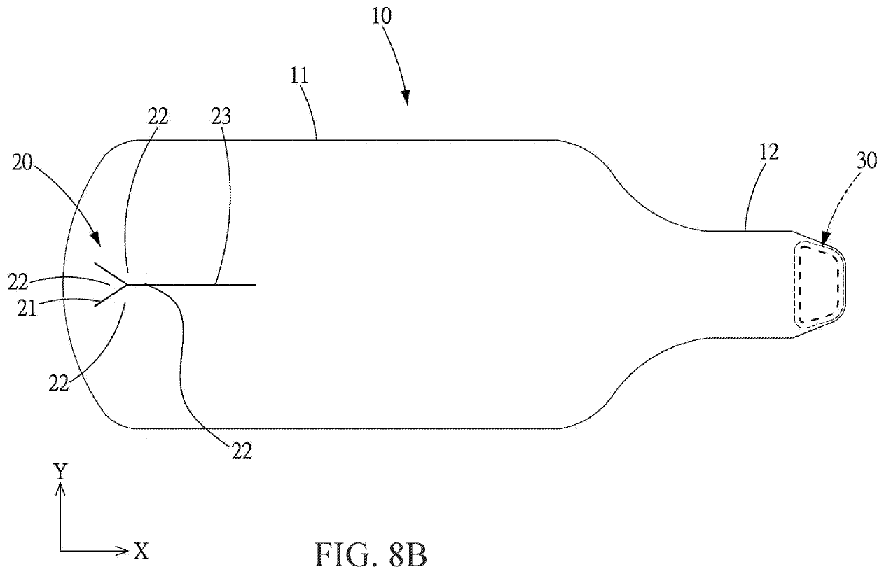

Referring to FIGS. 7 and 8, elastic bandages disclosed by a fifth preferred embodiment and a sixth embodiment of the present invention have structures similar to those in the foregoing embodiments. The opening 21 of the first fixing portion 20 is changed to an X shape, causing the generation of four movable pieces 22 on the first fixing portion 20. As compared with the fifth preferred embodiment, the first fixing portion 20 that is in the sixth preferred embodiment and similar to those in the second preferred embodiment and the fourth preferred embodiment, further has the tangent 23 extending along the X-axis direction, where the tangent has one end connected to the center of the opening 21. In addition, the tangent 23 further divides the four movable pieces 22 into five pieces, each resembling a triangle. This not only increases the area of the opening 21 but also enhances the mobility of the movable pieces 22.

Referring to FIGS. 8A and 8B, elastic bandages disclosed by a seventh preferred embodiment and an eighth embodiment of the present invention have structures similar to those in the foregoing embodiments. In a seventh preferred embodiment, the opening 21 of the first fixing portion 20 is changed to a V shape and has the tangent 23 extending along the X-axis direction, with one end connected to the opening 21, allowing generation of two movable pieces 22 on the first fixing portion 20.

Further, in the eighth preferred embodiment, the opening 21 of the first fixing portion 20 is changed to A shape and has the tangent 23 extending along the X-axis direction, with one end connected to the opening 21, allowing generation of three movable pieces 22 on the first fixing portion 20.

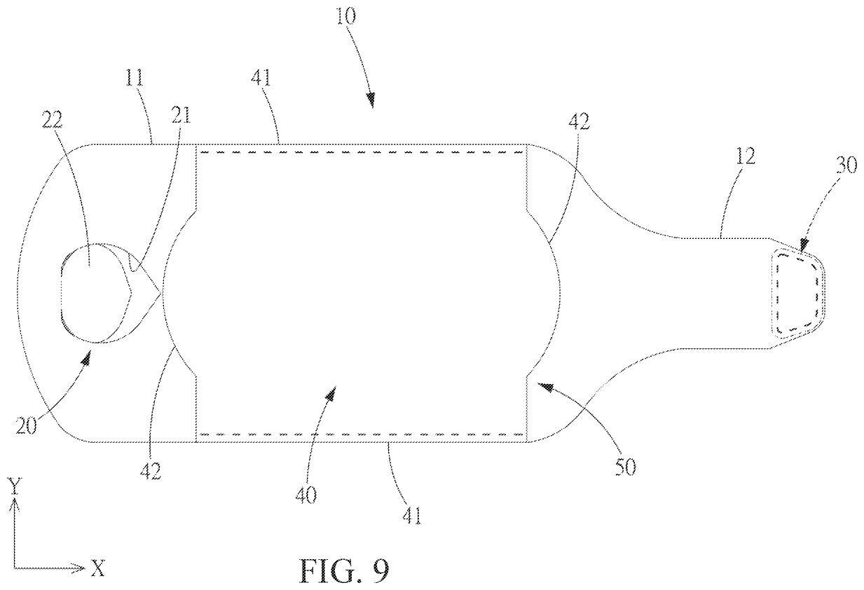

Referring to FIG. 9, the elastic bandage disclosed by a ninth preferred embodiment of the present invention has a structure similar to those of the elastic bandages in the foregoing preferred embodiments. In this embodiment, the elastic bandage further includes an accommodating portion 40. The accommodating portion 40, made of a non-woven fabric that is elastic and water-proof, is disposed on the wide portion 11 of the body 10. The accommodating portion 40 and the second fixing portion 30 are respectively disposed on two opposite sides of the body 10 along a Z-axis direction perpendicular to the X-axis direction and the Y-axis direction.

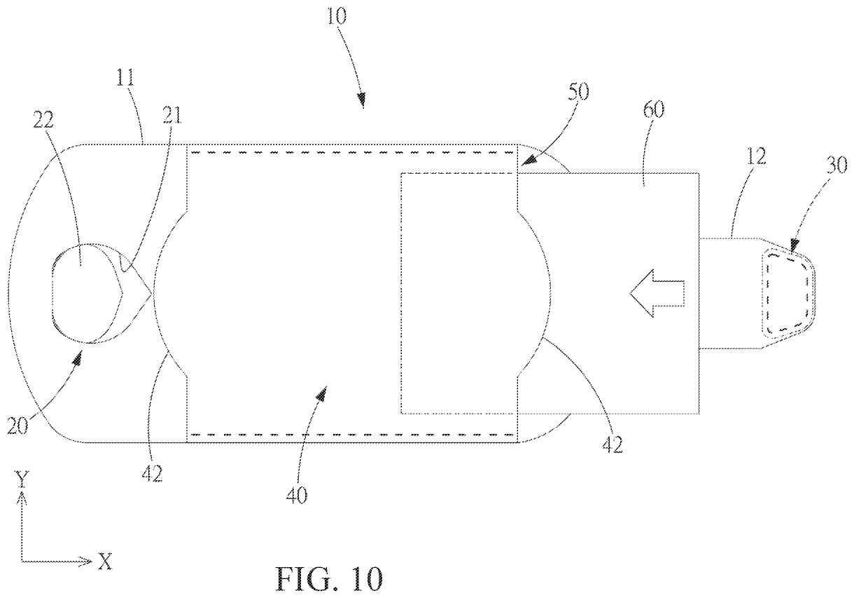

Two closed ends 41 are provided on two opposite sides of the accommodating portion 40 along the Y-axis direction and respectively connected to two opposite sides of the wide portion 11 along the Y-axis direction. Two open ends 42 are also provided on two opposite sides of the accommodating portion 40 along the X-axis direction, allowing for formation of an accommodating space 50 between the wide portion 11 of the body 10 and the accommodating portion 40, and the two open ends 42 both communicate with the accommodating space 50 The user can put a filling material 60 into the accommodating space 50 along any one of the two open ends 42 of the accommodating portion 40, as shown in FIG. 10.

In this way, when the user wraps the body 10 around the affected part 100 as shown in FIG. 3, the filling material 60 located in the accommodating space 50 can be adjacent to the affected part 100 and provide additional therapeutic effects. The filling material 60 may include but is not limited to items such as ice packs, hot packs, cold gels, shock-absorbing cushions (PU foam pads, high-density EVA foam pads), medicated patches, sterilized cloths, and tourniquets. These items can respectively provide the effects of cold compress, hot compress, cooling, joint protection, medication treatment, sterilization, and hemostasis.

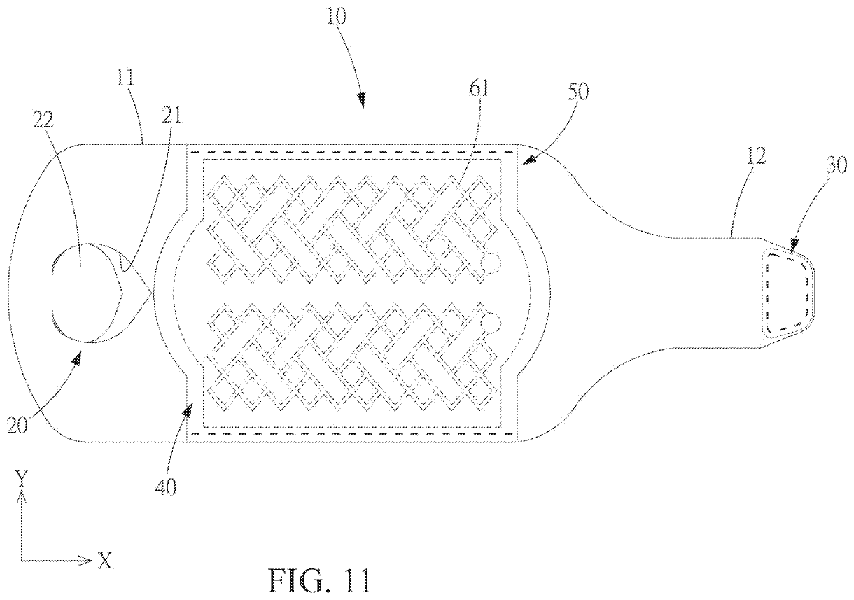

Referring to FIG. 11, the elastic bandage disclosed by a tenth preferred embodiment of the present invention has a structure similar to that of the elastic bandage in the ninth preferred embodiment. In this embodiment, the elastic bandage further includes an electrotherapy portion 61 disposed between the accommodating portion 40 and the wide portion 11 and electrically connected to a power supply (not shown in the figure), so as to provide a microcurrent for the affected part 100 when a user wraps the body 10 around the affected part 100, thus achieving the electrotherapy effect. The electrotherapy portion 61 in this embodiment can be fixed to a side of the accommodating portion 40 facing the wide portion 11. In another embodiment, the electrotherapy portion may serve as a filling material as in the ninth preferred embodiment to be detachably placed into the accommodating space.

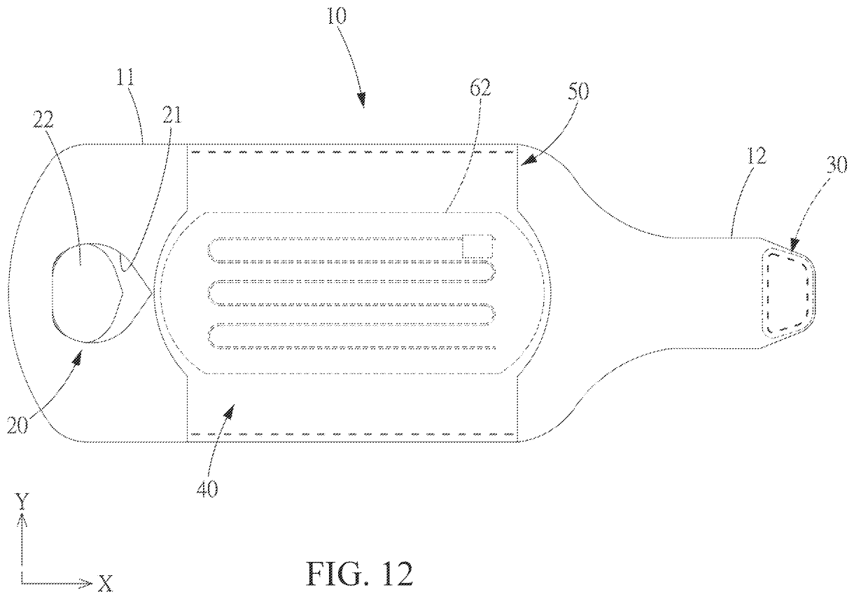

Referring to FIG. 12, the elastic bandage disclosed by an eleventh preferred embodiment of the present invention has a structure similar to that of the elastic bandage in the ninth preferred embodiment. In this embodiment, the elastic bandage further includes a thermotherapy portion 62 disposed between the accommodating portion 40 and the wide portion 11 and electrically connected to a power supply (not shown in the figure), so as to provide thermal energy for the affected part 100 when a user wraps the body 10 around the affected part 100, thus achieving the thermotherapy effect. The thermotherapy portion 62 in this embodiment can be fixed to a side of the accommodating portion 40 facing the wide portion 11. In another embodiment, the thermotherapy portion may serve as a filling material as in the ninth preferred embodiment to be detachably placed into the accommodating space.

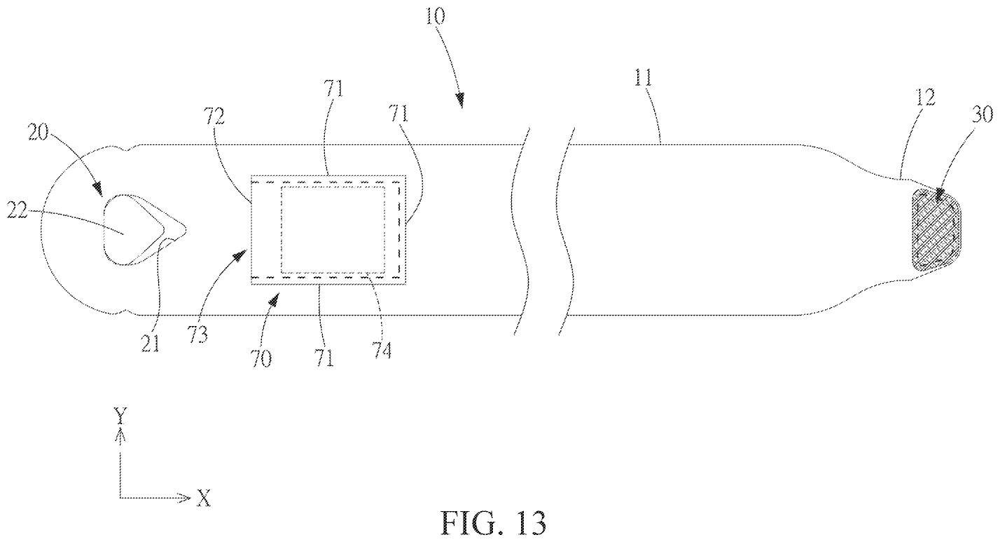

Referring to FIG. 13, the elastic bandage disclosed by a twelfth preferred embodiment of the present invention has a structure similar to those of the elastic bandages in the foregoing preferred embodiments. Along the X-axis direction, the wide portion 11 of the body 10 is longer than the narrow portion 12. In this embodiment, the elastic bandage further includes the accommodating portion 70 disposed on the wide portion 11 and adjacent to the first fixing portion 20. The accommodating portion 70, which is of a rectangular non-woven fabric structure, has one closed end 71 on each of two opposite sides along the Y-axis direction and a side along the X-axis direction, as well as an open end 72 on an opposite side along the X-axis direction, allowing for formation of an accommodating space 73 between the wide portion 11 of the body 10 and the accommodating portion 70, and the two open ends 72 both communicate with the accommodating space 73. The user can put a filling material 74 into the accommodating space 73 along the open end 72 of the accommodating portion 70. In this way, when the user wraps the body 10 around the affected part 100 as shown in FIG. 3, the filling material 74 located in the accommodating space 73 can be adjacent to the affected part 100 and provide additional medical functions. The filling material 74 may include but is not limited to items such as ice packs, hot packs, cold gels, shock-absorbing cushions (PU foam pads), medicated patches, sterilized cloths, and tourniquets. These items can respectively provide medical effects such as cold compress, hot compress, cooling, joint protection, medication treatment, sterilization, and hemostasis.

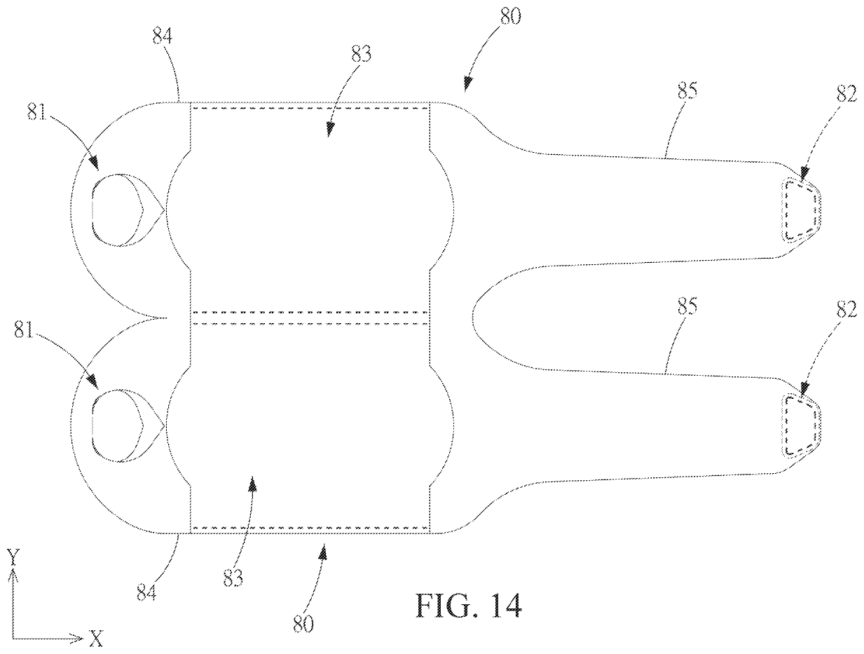

As shown in FIG. 14, a thirteenth preferred embodiment of the present invention provides an elastic bandage, including two bodies 80, two first fixing portions 81, two second fixing portions 82, and two accommodating portions 83. The body 80 includes a wide portion 84 and a narrow portion 85, and the two first fixing portions 81 and the two accommodating portions 83 are respectively disposed on the wide portions 84 of the two bodies 80. The two second fixing portions 82 are respectively disposed on the two narrow portions 85 of the body. The two bodies 80 are connected along the Y-axis direction, allowing adjacent sides of the two wide portions 84 to be connected along the Y-axis direction, and the two narrow portions 85 are spaced apart by a pre-determined distance. The elastic bandage described in this embodiment can wrap an affected part having a large area of the user, such as the front and back shoulders.

It should be noted that the present invention is not limited to the structural combination of the foregoing embodiments. In other embodiments, the first fixing portion in any arbitrary shapes, wide portion/narrow portion of any length ratio and width ratio, the presence and the position of the accommodating portion, any combination of the foregoing structures shall fall into the application scope of the present invention.

In conclusion, the elastic bandage provided by the present invention can effectively reduce the area of ineffective wrapping during bandaging by using wide and narrow portions in different sizes. In addition, the first fixing portion and the second fixing portion can improve the overall fixing strength of the elastic bandage. The design of the accommodating portion allows the elastic bandage to provide other medical functions besides bandaging. Therefore, the elastic bandage of the present invention can address the disadvantages of conventional elastic bandages and provide a simpler and more convenient use experience of bandages.

The foregoing embodiments are provided for illustrative purposes to demonstrate the technology and benefits of the present invention, and not intended to limit the scope of the present invention. Any person skilled in the art can modify and change the foregoing embodiments without departing from the technical principles and spirit of the present invention. Therefore, the protection scope of the present invention should be as defined in the claims below.

1. An elastic bandage, comprising:
a body comprising a wide portion and a narrow portion, wherein the wide portion is connected to the narrow portion along an X-axis direction, and along a Y-axis direction perpendicular to the X-axis direction, a width of the wide portion is greater than a width of the narrow portion;
a first fixing portion disposed on the wide portion of the body; and
a second fixing portion disposed on the narrow portion of the body; wherein
the first fixing portion and the second fixing portion are respectively located at two opposite ends of the body along the X-axis direction.

2. The elastic bandage according to claim 1, wherein the width of at least part of the body gradually narrows along the Y-axis direction from the wide portion to the narrow portion.

3. The elastic bandage according to claim 1, wherein the first fixing portion has an opening in a predetermined shape and a movable piece at least partially connected to the wide portion and corresponding to the opening, wherein the predetermined shape comprises water droplet shape, semi-circular shape, X shape, V shape, A shape, or irregular shape; and the movable piece is drivable to move with respect to the opening along a Z-axis direction perpendicular to the X-axis direction and the Y-axis direction.

4. The elastic bandage according to claim 3, wherein the first fixing portion further has a tangent with one end connected to the opening and the other end extending along the x-axis direction.

5. The elastic bandage according to claim 1, further comprising an accommodating portion disposed on the wide portion of the body, wherein the body is provided with an accommodating space formed between the accommodating portion and the wide portion.

6. The elastic bandage according to claim 5, wherein the accommodating portion and the second fixing portion are respectively disposed on two opposite sides of the body along a Z-axis direction perpendicular to the X-axis direction and the Y-axis direction.

7. The elastic bandage according to claim 5, wherein a side of the accommodating portion is provided with an open end communicating with the accommodating space and configured for a filling material to enter and exit the accommodating space along the open end.

8. The elastic bandage according to claim 5, wherein two closed ends are provided on two opposite sides of the accommodating portion along the Y-axis direction and respectively connected to two opposite sides of the wide portion along the Y-axis direction; and two opposite sides of the accommodating portion along the X-axis direction are provided with two open ends communicating with the accommodating space and configured for a filling material to enter and exit the accommodating space along any one of the two open ends.

9. The elastic bandage according to claim 5, further comprising an electrotherapy portion and/or a thermotherapy portion, wherein the electrotherapy portion is disposed between the accommodating portion and the wide portion and electrically connected to a power supply, so as to provide a microcurrent for an affected part when a user wraps the body around the affected part; and the thermotherapy portion is disposed between the accommodating portion and the wide portion and electrically connected to a power supply, so as to provide thermal energy for the affected part when the user wraps the body around the affected part.

10. An elastic bandage, comprising:
two bodies each comprising a wide portion and a narrow portion, wherein the wide portion and the narrow portion of each body are connected along an X-axis direction, and along a Y-axis direction perpendicular to the X-axis direction, a width of the wide portion is greater than a width of the narrow portion;
two first fixing portions respectively disposed on the two wide portions of the two bodies; and
two second fixing portions respectively disposed on the two narrow portions of the two bodies; wherein
the two wide portions of the two bodies are connected along the Y-axis direction;
two accommodating portions are connected along the Y-axis direction; and
the two narrow portions are spaced apart from each other by a predetermined distance along the Y-axis direction.
